(19) United States
(12) Patent Application Publication
(10) Pub. No.: US 2013/0052236 A1
(43) Pub. Date: Feb. 28, 2013

(54) COMPOSITE POLYLACTIC ACID/ALGINATE SURGICAL BARRIER

(71) Applicants: Joerg Tessmar, Regensberg (DE); Eva Esser, Regensberg (DE); Lukas Bluecher, Eurasberg (DE); Michael Milbocker, Holliston, MA (US)

(72) Inventors: Joerg Tessmar, Regensberg (DE); Eva Esser, Regensberg (DE); Lukas Bluecher, Eurasberg (DE); Michael Milbocker, Holliston, MA (US)

(73) Assignee: MAST BIOSURGERY, Zurich (CH)

(21) Appl. No.: 13/597,821

(22) Filed: Aug. 29, 2012

Related U.S. Application Data
(60) Provisional application No. 61/528,799, filed on Aug. 30, 2011.

(51) Int. Cl. A61K 9/00; A61F 13/00; A61K 38/44

(52) U.S. Cl. 424/400; 424/94.4; 427/2.31

(57) ABSTRACT

The present disclosure provides a medical assembly comprising a surgical barrier aspect comprising polylactic acid, and a hydrophilic mucoadhesive aspect, wherein the surgical barrier aspect is provided on a first side of the assembly and the mucoadhesive aspect is provided on a second side of the assembly. The disclosure also relates to a medical assembly comprising a surgical barrier aspect, a short term mucoadhesive aspect, an intermediate term protein polymerization adhesive aspect, and a long term tissue ingrowth implant localization aspect. The aforementioned medical assemblies may be provided as layered sheet structures. Also provided are methods for preparing a medical assembly.

### RELATED APPLICATIONS

This application claims the benefit of priority of U.S. provisional application No. 61/528,799, filed on Aug. 30, 2011, the contents of which are hereby incorporated by reference in their entirety.

### TECHNICAL FIELD

The present disclosure relates to implantable medical devices, and more specifically, to medical devices comprising mucoadhesive material and a surgical barrier material for implantation into living tissue of a subject and methods of making the same. More specifically, the present disclosure relates to anti-adhesion surgical barriers that are self-affixing to a surgical site.

### BACKGROUND

Medical treatments for the shortcomings or functional deficiencies of biological structures have long included implantable sheet structures for the reconstruction, reinforcement and sequestration of tissue defects. One common failure of such sheet structures is their propensity to migrate within the body after implantation. To mitigate this effect, such implants are commonly affixed to a surgical site with localization devices, such as suture, tacks, staples, and the like. Unfortunately, these localization devices are associated with acute and chronic adverse clinical outcomes. Acutely, these localization devices impinge on healthy tissue, and can cause localization of stress resulting in pressure necrosis and pain. Chronically, these localization devices become adhesion sites, and these adhesions can constrain normally differentially moving layers of tissue, thereby causing implant remodeling and pain.

The clinical need for localizing implantable sheet structures within a body comprises three aspects that are not met in all respects using the common implant localization means. In the first aspect, especially in laparoscopic surgeries, the implant is initially placed in one position and subsequently the surgeon needs to reposition the implant. Thus, there is a need for a tacky aspect to the implant, wherein the localization is sufficient to communicate to the surgeon the conformal aspect of the device when positioned at a first location, while maintaining the ability to reposition the implant at a second location for a short period of time, such as several minutes. In the second aspect, the implant needs to be localized sufficiently after final placement to resist migration subsequent to surgical closure and before the formation of healing tissue ingrowth, for example over a period of several hours. In the third aspect, the implant either is absorbed in situ or possesses a porosity that allows for tissue ingrowth. This is the most permanent form of implant localization, and functions on time periods of several days to many years. There are two competing biological processes concerned with implants. One process is directed to incorporating the implant into the structural and functional aspects of the surrounding tissue. The second process is directed to sequestering the implant from the structural and functional aspects of the surrounding tissue. Which of these two processes predominates depends largely on the biocompatibility of the implant.

An alternative to sutures, tacks, staples and the like are tissue adhesives. There are currently several types of commercially available tissue adhesives, made from synthetic and naturally existing components. Examples of tissue adhesives are cyanoacrylates, polyurethanes prepolymers, gelatin based adhesives, fibrin based adhesives, and collagen based adhesives. The most widely used synthetic adhesives are cyanoacrylates. However, cyanoacrylates are associated with a variety of adverse events such as inflammatory response, delayed healing, necrosis, and thrombosis, limit their internal use as adhesives. Gelatin based adhesives form a network via crosslinking by resorcinol and formaldehyde. As with the cyanoacrylates, gelatin based adhesives are associated with toxicity issues largely due to the formaldehyde. The primary disadvantage of these tissue adhesives is that they do not allow repositioning of the implant once the adhesive is applied.

Current solutions for the prevention of fibrous tissue growth on implanted devices have included the use of timed releasable drugs or molecules, such as heparin or other antibiotics and antithrombogenics. However, such methods typically have proven to be short-term solutions, that is, on the order of days to months. In addition, the use of such drugs is costly, may have deleterious effects on the immune system of the host animal, and may subject the host animal to the side effects of the drugs.

Accordingly, there is a need for sheet-form medical devices that can be easily implanted and possess sufficient localization to maintain the intended use of the surgeon. A need also exists for implantable adhesion barriers that resist migration within the body, and thus preserve the blocking function intended by the surgeon.

### BRIEF SUMMARY

Accordingly, it is an object of the present disclosure to provide a medical assembly comprising: a surgical barrier aspect, and a hydrophilic mucoadhesive aspect, wherein the surgical barrier aspect is provided on a first side of the assembly and the mucoadhesive aspect is provided on a second side of the assembly. The medical assemblies described herein are, in certain embodiments, suitable for substantially long implantation in a host animal. In certain embodiments, the surgical barrier aspect comprises polylactic acid. In other embodiments, the mucoadhesive aspect comprises alginate.

It is another object of the disclosure to provide a medical assembly possessing a mucoadhesive side comprising an alginate and metal salt, particularly an alkaline earth metal salt. The salt is preferably selected from the group consisting of calcium, strontium, barium, and magnesium, and less preferably from zinc, copper, or iron.

It is another object of the disclosure to provide the aforementioned medical assembly, wherein the barrier aspect is provided as a first layer within the medical assembly and the mucoadhesive aspect is provided as a second layer within the medical assembly, wherein and the layers are affixed together with a binding compound. For example, the binding compound can be polyethylene imine, cetrimid, or cationic phospholipids.

It is another object of the disclosure to provide a medical assembly suitable for substantially long-term implantation in a host animal, comprising a surgical barrier aspect comprising polylactic acid, a structural aspect, and a hydrophilic mucoadhesive, wherein the surgical barrier aspect is provided on a first side of the assembly, the mucoadhesive aspect is provided on a second side of the assembly, and the structural aspect is provided as a layer sandwiched between the barrier aspect and the mucoadhesive aspect. If the structural layer comprises alginate, it may in some embodiments be cross-linked with an alkaline earth metal salt to provide for greater durability after implantation into a mammalian body. In some embodiments, the hydrophilic mucoadhesive aspect comprises an alginate.

It is another object of the disclosure to condition the mucoadhesive aspect, which may comprises an alginate, with a compound containing an alcohol group or polyol, for example, glycerol. In some embodiments, an alcohol is preferred over water, since water can cause degradation of the polylactide barrier aspect.

It is another object of the disclosure to provide a medical assembly comprising a surgical barrier aspect, a mucoadhesive aspect, and an ingrowth aspect. For example, perforation through the medical assembly to provide tissue ingrowth and anchoring may be provided. Alternatively, a mesh may be affixed to the mucoadhesive aspect, for example a mesh comprising a strip localized on the perimeter of the medical assembly. In other embodiments, the tissue ingrowth aspect comprises both a perforation through the medical assembly and a mesh affixed to the mucoadhesive aspect.

It is another object of the disclosure to provide a medical assembly comprising a surgical barrier aspect, a mucoadhesive aspect and a protein polymerization aspect. For example a protein polymerization can comprise, in some embodiments, oxidized cellulose. Preferably the oxidized cellulose is woven of oxidized cellulose fibers.

It is another object of the disclosure to provide a medical assembly comprising: a surgical barrier aspect; a short term mucoadhesive aspect; an intermediate term protein polymerization adhesive aspect; and a long term tissue ingrowth implant localization aspect.

It should be recognized that all of the contemplated objects of the present disclosure are suitable and readily adaptable to the release of a therapeutic substance. Accordingly, in some embodiments, the medical assembly further comprises a releasable therapeutic substance.

It is another object of the disclosure to provide a process for medical assembly comprising cast layers of polylactic acid and alginate, wherein the alginate layer is cross-linked after casting, comprising: a) suspending an alkaline earth metal salt in a solution comprising alginate, b) casting the alginate solution on a surface, c) drying the cast alginate to form an alginate layer, d) contacting a weak acid with the alginate layer to cause the alkaline earth metal salt to dissolve within the alginate layer, thereby causing said alginate layer to crosslink. In certain embodiments, the alkaline earth metal salt is calcium citrate. In other embodiments, the weak acid is lactic acid.

It is another object of the disclosure to provide a method of forming a medical assembly, wherein a first layer is cast from solution at least a portion of which comprises polylactic acid, and a second layer is cast from solution at least a portion of which comprises alginate, wherein at least one solution contains a binding compound.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### DETAILED DESCRIPTION

Reference now will be made in detail to the embodiments of the present disclosure, one or more examples of which are set forth herein below. Each example is provided by way of explanation of the medical assembly of the present disclosure and is not a limitation. In fact, it will be apparent to those skilled in the art that various modifications and variations can be made to the teachings of the present disclosure without departing from the scope or spirit of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

This disclosure relates generally to implantable medical devices and more specifically to medical assemblies comprising a surgical barrier aspect and a hydrophilic mucoadhesive aspect for implantation into the living tissue of a host animal, and to methods of making the same. More specifically, the present disclosure provides anti-adhesion surgical barriers which are self-affixing to a surgical site. In particular embodiments, the medical assemblies disclosed herein are suitable for substantially long-term implantation in a host animal, particularly mammals, and more particularly humans. The medical assembly includes a surgical barrier aspect, at least a portion of which is formed of polylactic acid (PLA). In combination, a medical device suitable for preventing tissue adhesions on one side and self-affixing on the other side is obtained. The surgical barrier aspect, in certain embodiments, comprises polylactic acid (PLA).

Mucoadhesivity allows an implant to be placed securely at a tissue location, peeled up, and replaced securely at another tissue location. For example, mucoadhesivity allows a sheet comprising a mucoadhesive aspect to form reversible bonds with living tissue. Any mucoadhesive substance may be used in the mucoadhesive aspect. Furthermore, mucoadhesive substances may be chosen based on the desired properties of the medical assembly. For example, mucoadhesive substances may vary in properties, including but not limited to, their absorption time, degradability, biocompatibility, gelling temperature, etc. In certain embodiments, the mucoadhesive aspect comprises an alginate, cellulose, a cellulose derivative (e.g. oxidized regenerated cellulose, carboxymethyl cellulose), hyaluronic acid (particularly cross-linked hyaluronic acid), hyaluronic acid derivatives (sodium hyaluronate, ferric hyaluronate, chemically cross-linked hyaluronic acid), dextran, chitosan, carboxymethyl chitosan, gelatin/proteoglycan, poloxamer 407/pluronic F-127, polyethylene glycol/PLA, or any combination thereof.

In certain embodiments, the mucoadhesive aspect comprises an alginate. In more particular embodiments, the surgical barrier aspect comprises PLA and the mucoadhesive aspect comprises an alginate. While not being bound by any particular theory, the mucoadhesive property of alginates and alginates cross-linked with alkaline earth metals allows the medical device to self-affix at a surgical site. Alginates are hydrophilic marine biopolymers with a unique ability to form heat-stable gels that can develop and set at physiologically relevant temperatures. Alginates are a family of non-branched binary copolymers of 1-4 glycosidically linked β-D-mannuronic acid (M) and α-L-guluronic acid (G) residues. The relative amount of the two uronic acid monomers and their sequential arrangement along the polymer chain vary widely, depending on the origin of the alginate. Interestingly, the relative concentration of mannuronic acid and guluronic acid varies to suit biological functions. For example, the polyuronic acid structure varies as to whether it is employed in the stem or leaf botanical structures of seaweed.

The physical and chemical properties of alginates are dependent on how the G and M monomers are arranged in an alginate macromolecule. Alginate gel properties affected include pore size, stability and biodegradability, gel strength and elasticity. In particular, it is not just the relative content of G and M monomers that determine useful properties, but the sequence, position and local density (length of contiguous like blocks) of the G and M monomers are also important. For example, a macromolecule of GMMMMG possesses different characteristic from a macromolecule of MGGGGGM. Interestingly, gel biodegradability/strength and porosity are anti-correlated. High G content relative to M content alginate polymers are less biodegradable than high M content gels. Gels with high G content alginate generally have larger pore sizes and stronger gel strength relative to gels with high M content.

In addition to these monomeric considerations, alginate polymers can be cross-linked by inserting a divalent cation into the gel matrix, wherein the cation ionically links two negatively charged G blocks. Thus, in some embodiments, formation of multiple crosslinks among numerous alginate polymers provides stronger gel structures. The divalent cation is generally an alkaline earth metal, although other di or multivalent metal cations may be used. Accordingly, in certain embodiments, the mucoadhesive aspect comprises an alginate and an alkaline earth metal salt. Alkaline earth metals useful for the mucoadhesive aspect include, without limitation, calcium, strontium, and barium. In a particular embodiment, the alkaline earth salt is calcium citrate. In other embodiments, the mucoadhesive aspect comprises an alginate and a polyvalent metal salt that is not an alkaline earth metal, such as copper, nickel, zinc, lead, iron, manganese or cobalt.

The aforementioned ionic bonds between divalent cations and G blocks are relatively easily broken. For example, anionic scavenging molecules can leach the cations out of cross-linked alginate gels. The ease by which anions penetrate the gel matrix and subsequently the cations exit the matrix is determined to some extent by the molecular weight of the alginate polymers. The higher the molecular weight, the more difficult it is for cations to exit the gel matrix. Water also reduces crosslink density by swelling the alginate matrix, increasing its permeability, and causing the cations to diffuse out of the swelled gel. Alginate gels with fewer crosslinks degrade faster than those with more crosslinks.

Due to this diffusion limiting effect of higher molecular weight alginate polymers, higher molecular weight limits cation diffusion out of the cross-linked polymer, thereby limiting the loss of crosslinks. Consequently, these polymers degrade in vivo less quickly. On the other hand, this limitation on molecular kinetics also limits gelling time during the formation of cross-linked gels and affects macroscopic features of the gel, such as pore size. Alginate polymers typically have average molecular weights ranging from 2 to 2000 kD.

Insoluble alkaline earth salts of alginic acid such as calcium alginate or barium alginate (depending upon the gelling ion used) or insoluble transition metal salts of alginic acid such as for example salts of copper, nickel, zinc, lead, iron, manganese or cobalt can be manufactured with a known and predetermined content of alkaline earth ions by precipitation from the solutions.

Alginate gels useful in medical application are typically insoluble hydrogels comprising cross-linked alginate polymers containing large amounts of water. These hydrogels degrade in living tissue due to progressive loss of the gelling cations and a combination of hydrolysis and enzymatic lysis of polymer chains. Another degradation route involves exchange of the gelling multivalent cations such as calcium, barium and zinc with monovalent ions such as sodium and potassium supplied by the living tissue. Such ion exchanges results in alginate gels becoming alginate solutions that are then soluble in the fluids present in living tissue.

In certain embodiments, the mucoadhesive aspect comprises any of the mucoadhesive materials listed herein, and further comprises a plasticizer. Suitable plasticizers include, but are not limited to, polyethylene oxide, polypropylene oxide, glycols such as propylene glycol and polyethylene glycol, alcohols such as polyhydric alcohols including glycerin (also called glycerol) and sorbitol, glycerol esters such as glycerol triacetate, fatty acid triglycerides, naphthenic oils, aromatic oils, vegetable oils such as castor oil, low molecular weight rosin esters, polyterpenes, or any combinations thereof. In particular embodiments, the mucoadhesive aspect further comprises a compound comprising an alcohol group, particularly a polyol, such as glycerol.

When alginate gels are used in medical devices, it is advantageous in certain embodiments to provide a product that does not contain appreciable amounts of water. Water in a medical device, especially one which readily cultures microbes, is generally avoided. Furthermore, the device is generally less stable when water is present in the gel matrix. Accordingly, in certain embodiments, plasticizers can be used to provide a flexible, soft implant that can be readily shaped to meet clinical needs. Accordingly, in certain embodiments, the mucoadhesive aspect comprises an alginate and a plasticizer. Suitable plasticizers include, but are not limited to, polyethylene oxide, polypropylene oxide, glycols such as propylene glycol and polyethylene glycol, alcohols such as polyhydric alcohols including glycerin (also called glycerol) and sorbitol, glycerol esters such as glycerol triacetate, fatty acid triglycerides, naphthenic oils, aromatic oils, vegetable oils such as castor oil, low molecular weight rosin esters, polyterpenes, or any combinations thereof.

Alginic acid is substantially insoluble in water, but it is made soluble in water in the form of an alginate. Alginates are water-soluble when they are formed using monovalent alkali metals, such as sodium, potassium, lithium, magnesium, ammonium, and the substituted ammonium cations derived from lower amines, such as methyl amine, ethanol amine, diethanol amine, and triethanol amine. These salts are soluble in aqueous media above pH 4, and are converted back to alginic acid when the pH is lowered below about pH 4. Alternatively, water-insoluble alginates are formed if certain polyvalent cations, especially calcium, barium, strontium, zinc, copper(+2), aluminum, and mixtures thereof are present in the medium at appropriate concentrations.

While alginic acid is not soluble in polar solvents, alginates formed from polyvalent cations are predisposed to disperse in water in much the same was as are alginates formed from monovalent cations. While not being bound by any particular theory, it is believed that polyvalent alginates are prevented from dissolving completely in water is the crosslinking function of the polyvalent alkaline metals, in contrast to the way monovalent alginates readily dissolve in water. Because the alginic acid is cross-linked in polyvalent alginates, these alginates are of solid form and gel-like. In certain embodiments, these are useful properties for the medical assemblies described herein. Additionally, since the crosslinking polyvalent cations become mobile in an aqueous environment, crosslinks can be lost over time by migration of the cations out of the alginate matrix. Alternatively, these polyvalent cations can be replaced with monovalent cations normally present in living tissue. This feature of cross-linked alginates is advantageous when used in biodegradable implants, such as the present medical assemblies.

The dynamics established between the polar metal ions and hydrophobic alginic acid present within alginate and polar water found in living tissue establishes mucoadhesivity, wherein a sheet of cross-linked alginate forms reversible bonds with living tissue. Mucoadhesivity allows an implant to be placed securely at a tissue location, peeled up, and replaced securely at another tissue location. The degree of crosslinking, and similarly rate of dissolution, determines the duration of the mucoadhesive effect. All above properties possessed by cross-linked alginates are useful in the present disclosure, and their uses will be made explicit in the embodiments of the present disclosure.

As described in the BACKGROUND section of the present disclosure, the mucoadhesive functionality is one of three temporally differentiated localizing functions useful in implantable medical devices. While intermediate localization can be obtain by increasing the stability of the alginate layer, primarily through increase crosslink density, intermediate localization can also be obtained by denaturation of proteins present in fluids at the implant site. Converting aqueous proteins into sticky gels is accomplished through hydrophobic interactions. The denaturation of proteins is also a mechanism by which the body identifies a foreign body, and thus can result in the formation of reactive oxygen species and fibrosis.

Cations are hydrophilic, which makes cross-linked alginates also hydrophilic. However, alginates can be made more or less hydrophilic by grafting on alkylene oxides. Alginate may be reacted with alkylene oxides such as ethylene oxide and propylene oxide, to form a glycol alginate. When ethylene oxide is used the alginate is more hydrophilic, when propylene oxide is used the alginate is more hydrophobic. Thus the hydrophilicity of the alginate can be designed, and intermediate localization functionality can be obtained by tailoring the propylene oxide content of the alginate. Accordingly, in some embodiments, the mucoadhesive aspect comprises an alginate and an alkylene oxide, such as propylene oxide, ethylene oxide, or a combination thereof. More specifically, in certain embodiments, the mucoadhesive aspect comprises a glycol alginate.

The glycol is bonded to the alginate through carboxyl groups. This is a useful bond, since PLA comprises terminal carboxyl groups. Thus, in certain embodiments, glycol can be used to bond together layers of PLA and alginate.

Other modifications of alginates include the use of other plant derived compounds. For example, pectic substances, including pectins and pectates, and naturally occurring polysaccharides found in the roots, stems, leaves, and fruits of various plants, especially the peel of citrus fruits such as limes, lemons, grapefruits, and oranges. Pectins contain polymeric units derived from D-galacturonic acid. In certain embodiments, the mucoadhesive aspect comprises an alginate and a D-galacturonic acid containing compound, such as a pectic compound selected from the group consisting of pectins, pectates, naturally occurring polysaccharides, and combinations thereof.

Another example of plant-derived compounds useful in mucoadhesive aspect are carrageenans, which contain groups of sulfated galactans extracted from red seaweed. Carrageenans are linear chains of D-galactopyranosyl units joined with alternating D-glycosidic linkages. Carrageenans may, in part, be distinguished by the degree and position of sulfation. There are three main types of carrageenan: kappa carrageenan, iota carrageenan, and lambda carrageenan. Kappa carrageenans produce strong rigid gels while those made with iota products are flaccid and compliant. Lambda carrageenans do not gel in water. Accordingly, in certain embodiments, the mucoadhesive aspect comprises a sulfated galactan compound, such as carrageenan. More particularly, in certain embodiments, the mucoadhesive aspect comprises, in some embodiments, an alginate and a sulfated galactan, such as carrageenan.

In further embodiments, the mucoadhesive aspect further comprises a gelling agent. The same gelling agent can sometimes be used for alginate, pectic substances and carrageenan. Some gelling agents require an acid environment and are not as practical for implantable devices. Preferred gelling agents are those that provide a buffering effect or consume acid when the polyvalent cation is released. Calcium carbonate is an example of a gelling agent useful in forming gels of alginate, pectic substances and carrageenan within the pH range of living tissue.

Zinc is also useful as a source for gelling cations since it has been associated with wound healing and anti-microbial effects. On the other hand, Barium is a useful gelling agent in applications where radio-opacity is desired. Zinc and copper are useful for gelling and stabilizing gels against sterilization methods.

In certain embodiments, divalent cations are preferred. Examples are calcium(2+), barium(2+), strontium(2+), iron(2+), zinc(2+), copper(2+), and aluminum(3+). Of these calcium is most preferred. More particularly calcium carbonate, calcium disodium edetate, calcium oxalate, dicalcium phosphate, tricalcium phosphate, and tricalcium citrate are useful gelling agents.

In certain embodiments, it may be useful to minimize the molar amount of the gelling agent relative to the molar amount of the binding group, L-guluronic acid groups in the case of alginate. This can be achieved by using higher valence cations. For example, to fully saturate an alginate with crosslinks 1 mole of divalent cation is used for every 2 moles of L-guluronic acid. If a trivalent gelling agent is used, the amount of gelling agent need drops to a molar ratio of 1:3. Increasing the number of L-guluronic acid units associated with a single cation center tends increase the strength of the resulting gel. This option provides an additional degree of freedom in tailoring the mechanical characteristics of a gel to a particular application, rather than simply increasing the crosslink density or molar amount of cation.

Another approach to increasing crosslink density is to synthesize or select alginic acid with a higher weight percentage of guluronic acid or reducing the weight percent of mannuronic acid. Guluronic acid groups participate in crosslinking, and mannuronic acid groups do not. Alginic acid is a polymerization of guluronic and mannuronic acid groups, there for the distribution of guluronic acid group (G-blocks) relative to the position of mannuronic acid groups (M-blocks) impacts gel mechanical properties. In certain embodiments, the G-block content of the alginate is at least about 30%, preferably about 50% to about 90%, and more preferably about 60% to about 80%.

Other methods for adjusting crosslink density are more macroscopic in nature. For example, in some embodiments, the efficiency with which cation are released from a calcium salt can be enhanced by decreasing the pH. Compounds used to adjust gel pH are known as pH modifiers. A common pH modifier is gluconolactone, which must be present during gel formation to be effective. Also, keeping the gel in a mobile phase, i.e., with water present, extends crosslinking. Thus slower drying improves crosslinking efficiency. Conversely, drying the gel before all the calcium is released and reacted with the alginate reduces crosslink efficiency.

Leaving some guluronic blocks un-cross-linked may serve certain applications. For example, the monovalent form of silver may be bound in crosslink-deficient gels. Silver is known to possess anti-microbial functionality. When some of the guluronic blocks are left open, then the gel will be locally charged and will take up water more aggressively. The increased water uptake swells the gel, mobilizing those cations present. Thus, there is a three-fold effect, lower crosslink density, more water in the gel matrix, and faster rate of cation loss all contribute to faster implant dissolution in situ.

Many of the gels formed by cation crosslinks, especially those with high crosslink density, tend to be flaky when fully dehydrated. Commonly, a plasticizer is used to keep the gel in a malleable or elastic state. For example, glycerol is used in certain embodiments. However, increasing the ratio of non-crosslinking species can achieve the same result, especially those species that are in the liquid phase under the gelation conditions. These are called complementary binders or co-binders. For example, high M-block content alginates can serve as co-binders. Others known in the art are chitosan and its derivatives, hyaluronate, carboxymethyl cellulose, starch, modified starch, and glycol alginates. Co-binders employed as gel softeners are preferably water soluble. Accordingly, in certain embodiments, the mucoadhesive aspect further comprises a co-binder, such as any of the aforementioned co-binders.

Plasticizers serve two functions, they serve to soften and to make flexible the dehydrated gel, and they are also used to attract water and speed rehydration of the gel. Typical plasticizers are polyhydric alcohols such as glycerin, sorbitol, ethylene glycol, propylene glycol, and polyethylene glycol. Preferably, the plasticizer is non-toxic and does not affect the solubility of the gel-forming polymer. Plasticizers such as ethylene glycol and polyethylene glycol affect the solubility of alginate. Thus, in some embodiments it may be preferable to use the glycol alginate version, rather than the glycol alone.

In still other embodiments, an emollient can be used. Emollients used in certain embodiments include, for example, hyaluronan, lanolin oil; coconut oil; cocoa butter; olive oil; jojoba oils; castor oil; esters such as diisopropyl adipate, hydroxybenzoate esters, alkyl benzoate, iso-nonyl iso-nanoate diocyl adipate, octyl stearate, hexyl laurate, coco-caprylate, cetaryl isononanoate, isopropyl myristate, propylene glycol dicaprylate/dicaprate, octyldodecyl neopentanoate and propylene glycol isoceteth-3 acetate, decyl oleate, and caprylic/capric triglycerides; cyclomethicone; dimethicone; phenyltrimethicone; alkanes such as mineral oil, silicones such as dimethyl polysiloxane, and ethers such as dicapryl ether; polyoxypropylene butyl ethers, and polyoxypropylene cetyl ethers.

Some gel conditioners can also be used to increase the strength of association between a mucoadhesive alginate layer and an anti-adhesion PLA layer, in certain embodiments. For example, chitosan can render a side of an alginate layer anti-adhesive on its own, but employed between alginate and PLA layers can bind the two by dipole interactions due to its amino groups. Thus, in certain embodiments, the mucoadhesive aspect comprises chitosan.

Additionally, some gel conditioners can also be used to enhance the tissue adhesion of alginate or other mucoadhesive substances. In certain embodiments, phloroglucinol can be added in the aqueous phase during the gelation process to condition an alginate gel in much the same way glycerol is used. In certain embodiments, the phloroglucinol is in a polymeric form. For example, phloroglucinol can be reacted with diisocyanate in excess, such that there is minimal polymerization of the phloroglucinol. The resulting polymer can then be added can be added to fully dehydrated alginate gels to increase adhesion. Alternatively, phloroglucinol can be added to propylene carbonate and then reacted with stoichiometric amounts of diisocyanate to form phloroglucinol triisocyanate. The combination of the propylene carbonate and phloroglucinol triisocyanate can then be placed on the alginate layer and allowed to absorb in. The propylene carbonate can then be removed with a suitable solvent. Thus in certain embodiments, the mucoadhesive aspect further comprises phloroglucinol, more particularly, a polymeric phloroglucinol. In certain embodiments, polymeric forms of phloroglucinol type compounds may contain a plurality of from about 2 to about 500,000 monomer units of phloroglucinol, or of a derivative of phloroglucinol,

In other embodiments, phloroglucinol can be used to bond together PLA and alginate layers using haloperoxidase enzyme, an oxidizer, a halogen salt, and combinations thereof.

Another approach to increasing the adhesivity of the alginate layer is to add a mixed partial salt of a copolymer of maleic acid and an alkyl vinyl ether during the polymerization of the alginate, in which case an excess amount of divalent cation should be used. In certain embodiments, the mucoadhesive aspect comprises an alginate and a mixed partial salt of a copolymer of maleic acid or maleic anhydride and lower alkyl vinyl ether, more particularly, a copolymer of maleic acid or maleic anhydride and a lower alkyl vinyl ether having from 1 to about 5 carbon atoms. In more particular embodiments, the partial salt is formed during polymerization of the alginate.

Lower alkyl vinyl ether maleic polymers are readily obtained by copolymerizing a lower alkyl vinyl ether monomer, such as methyl vinyl ether, ethyl vinyl ether, divinyl ether, propyl vinyl ether, isobutyl vinyl ether, and the like, with maleic anhydride to yield the corresponding lower alkyl vinyl ether-maleic anhydride copolymer which is readily hydrolyzable to the acid copolymer.

In addition to enhancing the tissue adhesive, biologically functional additives can be used. One advantage of a PLA-alginate structure is that the PLA is largely hydrophobic and the alginate hydrophilic, allowing for addition of both hydrophilic and hydrophobic additives. Therapeutic additives that are useful in the devices of the present disclosure include antimicrobial agents such as iodine, sulfonamides, bisbiguanides, cetylpyridium chloride, domiphen bromide or phenolics; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, cimetidine, eugenol, or hydrocortisone; anesthetic agents such as lidocaine or benzocaine; anti-fungals; and aldehyde derivatives such as benzaldehyde; insulin; steroids; and anti-neoplastics.

It is recognized that in certain medical applications, combinations of these agents in the same device may be useful in order to obtain an optimal effect. Thus, for example, an antimicrobial and an anti-inflammatory agent may be combined in a single device to provide combined effectiveness. The one or more antimicrobial agents may be provided in such an amount of the composition that provides effective antimicrobial properties to the composition. The one or more antimicrobial agents, in certain embodiments, may be present in an amount about 0.0001% to about 2.0% by weight of the composition, preferably 0.001% to about 1.0% by weight, and more preferably from about 0.01% to about 0.5% by weight of the composition.

In certain embodiments of the aforementioned medical assemblies, the barrier aspect is provided as a first layer within the medical assembly and the mucoadhesive aspect is provided as a second layer within the medical assembly. Thus, the medical assemblies described herein have, in certain embodiments, a multi-layered sheet structure. The first and second layers may optionally be affixed together with a binding compound. For example, the binding compound can be polyethylene imine, cetrimid, or cationic phospholipids.

In still other embodiments, the medical assembly comprises a surgical barrier aspect at least a portion of which is formed of polylactic acid; a structural aspect; and a hydrophilic mucoadhesive aspect, wherein the surgical barrier aspect is provided on a first side of the assembly and the mucoadhesive aspect is provided on a second side of the assembly, and the structural aspect is disposed there between. More particularly, the barrier aspect, structural aspect and mucoadhesive aspects are, in certain embodiments, formed as layers. In certain embodiments, the mucoadhesive aspect comprises an alginate, and the structural aspect comprises and alginate and a transition metal salt, as described above. In other embodiments, the mucoadhesive aspect comprises an alginate and a transition metal salt, and the structural aspect comprises a transition metal salt, wherein the wherein the weight fraction of alkaline earth metal salt in the structural aspect is greater than the weight fraction of alkaline earth metal in the mucoadhesive aspect.

In certain embodiments, the medical assembly further comprises an ingrowth aspect. For example, the ingrowth aspect comprises, in certain embodiments, at least one perforation through the medical assembly. The at least one perforation may have, by way of example, an area of from about 1 mm² to about 4 mm². In other embodiments, the ingrowth aspect comprises a mesh affixed to the mucoadhesive aspect comprising a strip on the perimeter of said medical assembly. In further embodiments, the ingrowth aspect comprises a perforation through the medical assembly and the mesh affixed to the mucoadhesive aspect.

The prior specific description concerned surgical barrier assemblies of the present disclosure with a mucoadhesive layer for localizing the device in a mammalian body. In other embodiments, hybrid mucoadhesive-polymerization aspects are provided.

As mentioned previously the mucoadhesive aspect, such as an alginate, is a short-acting adhesive, which provides for securely repositioning sheet implants in situ. Polymerization of aqueous proteins present in a living mammal provides for intermediate localization of an implant. Thus, in certain embodiments, the mucoadhesive aspect further comprises a protein polymerization aspect. An example of this effect is the use of oxidized cellulose in hemostasis. In an embodiment, cellulose and alginate are combined to provide an adhesive layer to an absorbable implant with both mucoadhesive and protein polymerization functionality.

Alginate can be cross-linked through a polycarboxylic acid ester bond to cellulose. Preferably, the alginate is cationic. The crosslinker must be biocompatible, for example, a polycarboxylic acid such as citric acid, maleic acid, itaconic acid, succinic acid, trans-aconitic acid, cis-aconitic acid, tricarbalyllylic acid, 1,2,3,-benzenetricarboxylic acid, 1,2,4-benzenetricarboxylic acid, 1,2,3,4-butanetetracarboxylic acid, 1,2,3,4-cyclobutanetetracarboxylic acid, all-cis-1,2,3,4-cyclopentanetetracarboxylic acid, tetrahydrofuran-2,3,4,5-tetracarboxylic acid, 1,2,4,5-benzenetetracarboxylic acid, all-cis-1,2,3,4,5,6-cyclohexacarboxylic acid, mellitic acid, or polymaleic acid.

In some methods of manufacture it can be advantageous to catalyze the polymerization reaction with an acid catalyst. The acid catalyst may be lithium hydrogen phosphate, sodium hydrogen phosphate, potassium hydrogen phosphate, lithium hydrogen phosphate, sodium hydrogen phosphate, potassium hydrogen phosphate, sodium phosphate, sodium carbonate, calcium hydrogen phosphate, sodium hypophosphite, or sodium phosphite.

As noted above, the polycarboxylic acid may be maleic acid. The attachment of alginate to maleate esters of cellulose proceeds through formation of a polycarboxylic acid ester bond with both alginate and cellulose. Formation of this bond may form to link the alginate to the cellulose and to also form crosslinks between neighboring cellulose molecular chains and alginate chains in an aqueous phase. The polymerizing solution can be poured onto a glass plate to form a sheet of covalently polymerized alginate and cellulose. This same chemical reaction may be performed on any polysaccharide possessing hydroxyl groups which may form an ester bond to a polycarboxylic acid.

This crosslinking scheme is also useful in crosslinking alginate on its own, or between mixtures of different alginates. The crosslinking of the alginates is by action of a crosslinking agent to provide covalent bonding, through the crosslinking agent, from the carboxylic acid groups of the uronic acid of one alginate unit to the carboxylic acid group of the uronic acid of another alginate unit. Crosslinking contributes to gel stability and strength when the polymerization is between alginate units from different alginate chains. However, crosslinking may also occur between alginate units of the same chain.

As explained in the BACKGROUND section, there is a need in medical devices for three features: a short-term tacky aspect, an intermediate term adhesive aspect, and a long term ingrowth aspect. Regarding the short term tacky aspect, the localization is sufficient to communicate to the surgeon the conformal aspect of the device when positioned at a first location, while maintaining the ability to reposition the implant at a second location. Consequently, the adhesivity of the implant needs to be high under shear stress and relatively less adhesive under peel stress. In short, the device needs to be localized, and then the localization reversed relatively easily without losing adhesivity under multiple instances of repositioning. This first aspect of adhesivity needs to possess an effective time period of several minutes.

In the second aspect, the implant needs to be localized sufficiently after final placement to resist migration subsequent to surgical closure and before the formation of healing tissue ingrowth. This can be achieved by a number of mechanisms which act over the time period of several hours. Exemplary mechanisms include denaturation/polymerization of proteins contained in bodily fluids present at the surgical site, hydrophobic bonding effects, ionic affinity, and combinations of these, for example mucoadhesivity. The mechanisms behind mucoadhesion have not yet been fully elucidated, but a generally accepted theory is that close contact must first be established between the mucoadhesive agent and tissue, followed by interpenetration of the mucoadhesive polymer and the mucin and finishing with the formation of entanglements and chemical bonds between the macromolecules. The chemical bonds formed are mediated by hydrogen bonding groups contained in the mucoadhesive.

In the third aspect, the implant either is absorbed in situ or possesses a porosity that allows for tissue ingrowth. This is the most permanent form of implant localization, and functions on time periods of several days to many years. There are two competing biological processes concerned with implants. One process is directed to incorporating the implant into the structural and functional aspects of the surrounding tissue. The second process is directed to sequestering the implant from the structural and functional aspects of the surrounding tissue. Which of these two processes predominates depends largely on the biocompatibility of the implant.

Thus in certain embodiments, the present disclosure provides a medical assembly comprising a surgical barrier aspect, a short term mucoadhesive aspect, an intermediate term protein polymerization aspect, and a long term tissue ingrowth implant localization aspect. The surgical barrier aspect, mucoadhesive aspect, protein polymerization aspect, and tissue ingrowth aspect can be any as described herein.

The present disclosure also provides processes for preparing any of the aforementioned medical assemblies. In certain embodiments, the process comprises casting a layer comprising the surgical barrier aspect and casting a layer comprising the mucoadhesive aspect. In embodiments comprising a structural aspect, the method further provides the step of casting the structural layer. In certain embodiments, a process for forming a medical assembly comprises casting layers of polylactic acid and alginate, wherein the alginate layer is cross-linked after casting, comprising: a) suspending a low solubility alkaline earth metal salt in a solution comprising alginate, b) casting the alginate solution on a surface, c) drying the cast alginate to form an alginate layer, d) contacting a weak acid with the alginate layer to cause the low solubility alkaline earth metal salt to dissolve within the alginate layer, thereby causing the alginate layer to crosslink. The resulting medical assembly comprises a cast layer of PLA and a cast layer of alginate, wherein the alginate portion is cross-linked. In certain embodiments, the alkaline metal salt is calcium citrate, and the weak acid is citric acid. In particular embodiments, the contacting step comprises spraying or spritzing the weak acid onto the alginate layer.

In another embodiment, a method of forming a medical assembly comprises casting a solution comprising the surgical barrier material in a first layer, and casting a solution comprising the mucoadhesive material into a second layer, wherein at least one of the solutions further comprises a binding compound. In some embodiments, the solution comprising the surgical barrier material comprises the binding compound, while in other embodiments, the solutions comprising the mucoadhesive material comprises the binding compound. In still further embodiments, both solutions comprise the binding compound. The binding compound can be any binding compound, and particular embodiments, is a binding compound as described above. For example, in some embodiments, the method comprises casting a solution comprising polylactic acid in a first layer, and casting a solution comprising alginate into a second layer, wherein at least one of the solutions further comprises a binding compound.

In another embodiment, a method of forming a medical assembly comprises casting a solution comprising polylactic acid in a first layer, and casting a solution comprising alginate into a second layer, wherein at least one of the solutions further comprises a binding compound.

The present disclosure further provides methods of preventing surgical adhesions or promoting healing in a surgical site comprising administering a medical assembly as described herein to a surgical site. In particular embodiments, the surgery is in the interperitoneal cavity of a subject. In other embodiments, the surgery is a laproscopic surgery.

The composition of the present disclosure may be free of substantially free of any optional or selected ingredients described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in surgical barriers.

As used herein, the term “about” should be construed to refer to both of the numbers specified in any range. Any reference to a range should be considered as providing support for any subset within that range.

Examples are provided to illustrate some embodiments of the medical assemblies and processes of the present disclosure but should not be interpreted as any limitation thereon. Other embodiments within the scope of the claims herein will be apparent to one skilled in the art from. It is intended that the specification, together with the examples, be considered to be exemplary only, with the scope and spirit of the disclosure being indicated by the claims which follow the examples.

### EXAMPLES

The alginates used in these examples are PROTANAL LF 10/60 (FMC BioPolymer, Philadelphia, Pa.) and PROTANAL LF 10/60 (FT FMC BioPolymer, Philadelphia, Pa.). A lipophilic polymer: used is PLA LR 708 (Boehringer Ingelheim, Frankfurt, Germany). Crosslinking agents for the alginate portions are calcium citrate tetrahydrate (Sigma Aldrich, St. Louis, Mo.) and DL-lactic acid (Fluka, St. Louis, Mo.). The softener and moisturizer for film cohesion is glycerin 87% (AppliChem, Manchester, England). Film cohesion mediating substances include polyethylene imine (PEI) (Sigma Aldrich, St. Louis, Mo.), Cetrimid (Sigma Aldrich, St. Louis, Mo.), and cationic phospholipids, like lipofectamine and. EDOPC (o-ethyldioleoylphosphatidylcholinium (Sigma Aldrich, St. Louis, Mo.). Solvents used are dichloromethane and Millipore water (Sigma Aldrich, St. Louis, Mo.).

### Example 1

Anti-Adhesion Device Comprised of Alginate and Glycerol; Alginate, Glycerol and Calcium Citrate Tetrahydrate and PLA:

Into a beaker equipped with a magnetic stirrer was introduced 1.8 g PLA LR 708 dissolved in 43 ml dichloromethane and stirrer for 12 hours. This solution was cast on a glass slide with an Erichsen coatmaster 509 MC (Erichsen, Hemer, Germany), with a gap clearance of 250 μm and at a speed of 5 mm/s. The formed layer was slowly hardened by convective evaporation at room temperature to dryness. For the second layer, 4 g alginate LF 10/60 and 0.4 g glycerol were dissolved in 95.6 g millipore water using a magnetic stirrer. To 25 ml of this solution was added 750 mg calcium citrate tetrahydrate and aggressively stirred until dissolves. This solution was cast over the dry PLA layer, with a gap clearance of 700 μm and at a speed of 5 mm/s. After drying the resulting composite film consisting of two layers, the surface was spritzed with 0.5 ml lactic acid to dissolve the calcium citrate and initiated crosslinking the alginate. After convective drying a third layer was cast using a solution of 4 g alginate LF 10/60 FT and 0.4 g glycerol dissolved in 95.6 g millipore water. This solution was cast over the dry cross-linked alginate layer with a gap clearance of 700 μm and at a speed of 5 mm/s. After drying the film consisting of three layers can be peeled off the glass slide. After drying the resulting composite film consisting of three layers, the product was peeled off the glass slide.

### Example 2

Anti-Adhesion Device Comprised of PLA, Alginate and Glycerol:

Into a beaker equipped with a magnetic stirrer was introduced 1.8 g PLA LR 708 dissolved in 43 ml dichloromethane and stirrer for 12 hours. This solution was cast on a glass slide with an Erichsen coatmaster 509 MC (Erichsen, Hemer, Germany), with a gap clearance of 250 μm and at a speed of 5 mm/s. The formed layer was slowly hardened by convective evaporation at room temperature to dryness. For the second layer, 6 g alginate LF 10/60 and 0.6 g glycerol were dissolved in 93.4 g millipore water using a magnetic stirrer. This solution was cast over the dry PLA layer, with a gap clearance of 700 μm and at a speed of 5 mm/s. After drying the resulting composite film consisting of two layers, the product was peeled off the glass slide.

### Example 3

Anti-Adhesion Device Comprised of Alginate and Glycerol; and PLA:

Into a beaker equipped with a magnetic stirrer was introduced 6 g alginate LF 10/60 and 0.6 g glycerol dissolved in 93.4 g millipore water using a magnetic stirrer for 12 hours. This solution was cast on a glass slide with an Erichsen coatmaster 509 MC (Erichsen, Hemer, Germany), with a gap clearance of 250 μm and at a speed of 5 mm/s. The formed layer was slowly hardened by convective evaporation at room temperature to dryness. For the second layer, into a beaker equipped with a magnetic stirrer was introduced 1.8 g PLA LR 708 dissolved in 43 ml dichloromethane and stirred for 12 hours. This solution was cast over the dry alginate layer, with a gap clearance of 700 μm and at a speed of 5 mm/s. After drying the resulting composite film consisting of two layers, the product was peeled off the glass slide.

### Example 4

Anti-Adhesion Device Comprised of PLA, PEI, Alginate and Glycerol:

Into a beaker equipped with a magnetic stirrer was introduced 1.8 g PLA LR 708 and 0.036 g PEI was dissolved in 43 ml dichloromethane and stirrer for 12 hours. This solution was cast on a glass slide with an Erichsen coatmaster 509 MC (Erichsen, Hemer, Germany), with a gap clearance of 250 μm and at a speed of 5 mm/s. The formed layer was slowly hardened by convective evaporation at room temperature to dryness. For the second layer, 6 g alginate LF 10/60 and 0.6 g glycerol were dissolved in 93.4 g millipore water using a magnetic stirrer. This solution was cast over the dry PLA layer, with a gap clearance of 700 μm and at a speed of 5 mm/s. After drying the resulting composite film consisting of two layers, the product was peeled off the glass slide.

### Example 5

Anti-Adhesion Device Comprised of Alginate and Glycerol; PLA and PEI:

Into a beaker 6 g alginate LF 10/60 and 0.6 g glycerol were dissolved in 93.4 g millipore water using a magnetic stirrer. This solution was cast on a glass slide with an Erichsen coatmaster 509 MC (Erichsen, Hemer, Germany), with a gap clearance of 250 μm and at a speed of 5 mm/s. The formed layer was slowly hardened by convective evaporation at room temperature to dryness. For the second layer, into a beaker equipped with a magnetic stirrer was introduced 1.8 g PLA LR 708 and 0.036 g PEI was dissolved in 43 ml dichloromethane and stirrer for 12 hours. This solution was cast over the dry alginate layer, with a gap clearance of 700 μm and at a speed of 5 mm/s. After drying the resulting composite film consisting of two layers, the product was peeled off the glass slide.

### Example 6

Adhesive Testing:

Prototype composite structures of alginate and PLA were manufactured according to EXAMPLES 1-5. The adhesiveness was measured in shear and peel. Cutlets of fresh chicken breast were purchased and sliced into 3 cm cubes and affixed to a localized platform. The meat was kept well hydrated with physiologic saline solution at 22° C. Test articles were cut to 1 cm strips, yielding in most cases 14 strips. One test article yielded only 7 strips, which were cut in half to yield 14 shorter length strips. Both sides of the test article were tested with respect to shear and peel strength. Shear was measured by placing the strip on the 3 cm cube of meat and pulling horizontally to the surface. Thus these measurements yield a force per unit area (3 cm2). Peel was measured by pulling orthogonal to the cube surface, thus yielding a force per unit width (1 cm). Shear and peel were tested immediately upon contact and after 5 minutes. The tissue surface was kept moist to replicate normal surgical conditions (wet to touch), but no standing water. The same strip was used for the immediate measurement and the subsequent 5 minute measurement. Thus 4 strips, shear top side, shear bottom side, peel top side and peel bottom side were used, and four tissue cubes were used for each run. Three runs were performed giving a total of 12 strips per prototype. Outliers were discarded, and the 2 extra strips were used for additional runs as needed.

An Instron Mini 55 was used to record force and the crosshead speed was 0.1 cm/sec. The load cell limit was 200 g with an accuracy of +/−0.1 g. We expect an initial high resistance to shear (static friction) followed by a lower resistance to shear (kinetic friction). This information was obtained from shear results only, since peel did not exhibit a difference. The static and kinetic values were means based on changes in the Force vs Time slope.

### Shear and Peel Tests:

All measurement rounded to nearest gram. EXAMPLE 1 shear and peel results are depicted in Table 1.

### Alginate+Glycerol 1:1 (up)

### Alginate+Glycerol+Ca^{LT}

PLA (down)

TABLE 1

| | Immediate | | 5 minute delay | |
|---|---|---|---|---|
| N = 3 | Static (kinetic) | | Static (kinetic) | |
| Shear (g/9 cm2) | | | | |
| down | 42 +/− 8 | (26 +/− 10) | 19 +/− 5 | (15 +/− 5) |
| up | 8 +/− 3 | (1) | 2 +/− 2 | (1) |
| Peel (g/cm) | | | | |

| | | |
|---|---|---|
| down | 8 +/− 2 | 3 +/− 2 |
| up | 1 (down layer | 1 |
| | delaminated) | |

Sides in contact with tissue:
Down = side under with cut in top right
Up = side upper with cut in top right

EXAMPLE 2 results are depicted in Table 2.

with PLA (up) and Alginate+glycerol 1:1 (down) are depicted in Table 2.

TABLE 2

| | Immediate | | 5 minute delay | |
|---|---|---|---|---|
| N = 3 | Static (kinetic) | | Static (kinetic) | |
| Shear (g/9 cm2) | | | | |
| down | 78 +/− 10 | (45 +/− 17) | 78 +/− 12 | (47 +/− 19) |
| up | 16 +/− 5 | (1) | 9 +/− 5 | (1) |
| Peel (g/cm) | | | | |

| | | |
|---|---|---|
| down | 22 +/− 7 | 18 +/− 5 |
| up | 1 | 1 |

EXAMPLE 3 results are depicted in Table 3.

Alginate+glycerol 1:1 (up)
PLA (down)

TABLE 3

| | Immediate | 5 minute delay |
|---|---|---|
| N = 3 | Static (kinetic) | Static (kinetic) |
| Shear (g/9 cm2) | | |
| Down | [symbol] 151 +/− 23 | [symbol] 122 +/− 18 |
| up | 49 +/− 12 (17 +/− 6) | 12 +/− 6 (5 +/− 2) |
| Peel (g/cm) | | |
| down | 13 +/− 5 | 6 +/− 5 |
| up | 1 | 1 |

[symbol]

Indicates sample broke before motion was detected

EXAMPLE 4 results are depicted in Table 4.

### PLA+PEI (2%) (up)

Alginate+glycerol (10%) (down)

TABLE 4

| | Immediate | | 5 minute delay | |
|---|---|---|---|---|
| N = 3 | Static (kinetic) | | Static (kinetic) | |
| Shear (g/9 cm2) | | | | |
| Down | 82 +/− 26 | (55 +/− 23) | 42 +/− 16 | (34 +/− 12) |
| up | 28 +/− 11 | (12 +/− 5) | 12 +/− 6 | (6 +/− 2) |
| Peel (g/cm) | | | | |

| | | |
|---|---|---|
| down | 10 +/− 4 | 5 +/− 3 |
| up | 1 | 1 |

EXAMPLE 5 shear and peel results are depicted in Table 5.

Alginate+glycerol (10%) (up)
PLA+PEI (2%) (down)

TABLE 5

| | Immediate | 5 minute delay |
|---|---|---|
| N = 3 | Static (kinetic) | Static (kinetic) |
| Shear (g/9 cm2) | | |
| Down | 55 +/− 31 (26 +/− 13) | 23 +/− 17 (12 +/− 7) |
| up | 71 +/− 20 (55 +/− 24) | 27 +/− 12 (22 +/− 7) |
| Peel (g/cm) | | |
| down | 7 +/− 3 | 3 +/− 3 |
| up | 23 +/− 7 | 5 +/− 5 |

What is claimed is:

1. A medical assembly comprising:
a surgical barrier aspect comprising polylactic acid, and
a hydrophilic mucoadhesive aspect,
wherein the surgical barrier aspect is provided on a first side of the assembly and the mucoadhesive aspect is provided on a second side of the assembly.

2. The medical assembly of claim 1, wherein the mucoadhesive aspect comprises an alginate.

3. The medical assembly of claim 2, wherein the mucoadhesive aspect further comprises an alkaline earth metal salt.

4. The medical assembly of claim 3, wherein the alkaline earth metal salt is selected from the group consisting of calcium, strontium, or barium.

5. The medical assembly of claim 1, wherein the mucoadhesive aspect further comprises a D-galacturonic acid-containing substance.

6. The medical assembly of claim 5, wherein the D-galacturonic acid-containing substance comprises a pectic compound selected from the group consisting of pectins, pectates, naturally occurring polysaccharides, and any combination thereof.

7. The medical assembly of claim 1, wherein the mucoadhesive aspect further comprises sulfated galactans.

8. The medical assembly of claim 6, wherein the sulfated galactan is carrageenan.

9. The medical assembly of claim 1, wherein the mucoadhesive side further comprises phloroglucinol.

10. The medical assembly of claim 9 wherein the phloroglucinol is reacted with a diisocyanate such that there is minimal polymerization of the phloroglucinol.

11. The medical assembly of claim 3, wherein the mucoadhesive aspect further comprises a partial salt of a copolymer of maleic acid and an alkyl vinyl ether.

12. The medical assembly of claim 1, wherein the barrier aspect forms a first layer and the mucoadhesive aspect forms a second layer, wherein the first layer and the second layer are affixed together with a binding compound.

13. The medical assembly of claim 12, wherein the binding compound comprises polyethylene imine, cetrimid, cationic phospholipids, or a combination thereof.

14. The medical assembly of claim 12, wherein the binding compound comprises a combination of phloroglucinol, haloperoxidase enzyme, an oxidizer, and a halogen salt.

15. The medical assembly of claim 1 wherein the mucoadhesive aspect further comprises an alcohol.

16. The medical assembly of claim 15, wherein the alcohol is glycerol.

17. The medical assembly of claim 1, further comprising an ingrowth aspect.

18. The medical assembly of claim 17, wherein the ingrowth aspect comprises at least one perforation through the medical assembly.

19. The medical assembly of claim 28 wherein the at least one perforation has an area of from about 1 mm² to about 4 mm².

20. The medical assembly of claim 17, wherein the ingrowth aspect comprises a mesh affixed to the mucoadhesive aspect comprising a strip on the perimeter of the medical assembly.

21. The medical assembly of claim 20, wherein the second side providing the mucoadhesive aspect further comprises a protein polymerization aspect.

22. The medical assembly of claim 1, wherein the mucoadhesive aspect comprises an alginate, cellulose, a cellulose derivative, hyaluronic acid, hyaluronic acid derivatives, dextran, chitosan, carboxymethyl chitosan, gelatin/proteoglycan, poloxamer 407/pluronic F-127, polyethylene glycol/PLA, or any combination thereof.

23. A medical assembly comprising: a surgical barrier aspect comprising polylactic acid, a structural aspect, and a hydrophilic mucoadhesive aspect, wherein the surgical barrier aspect is provided on a first side of the assembly and the mucoadhesive aspect is provided on a second side of the assembly, and the structural aspect is disposed there between.

24. The medical assembly of claim 23, wherein the structural aspect comprises alginate and an alkaline earth metal salt.

25. The medical assembly of claim 24, wherein the mucoadhesive aspect comprises alginate and an alkaline earth metal salt, wherein the weight fraction of alkaline earth metal salt in the structural aspect is greater than the weight fraction of alkaline earth metal in the mucoadhesive aspect.

26. The medical assembly of claim 23 wherein the mucoadhesive aspect further comprises an alcohol.

27. The medical assembly of claim 26, wherein the alcohol is glycerol.

28. The medical assembly of claim 1, wherein the second side providing the mucoadhesive aspect further comprises a protein polymerization aspect.

29. The medical assembly of claim 28, wherein the protein polymerization aspect comprises oxidized cellulose.

30. The medical assembly of claim 29, wherein the protein polymerization aspect comprises woven oxidized cellulose fibers.

31. A medical assembly comprising:
a surgical barrier aspect;
a short term mucoadhesive aspect;
an intermediate term protein polymerization adhesive aspect; and
a long term tissue ingrowth implant localization aspect.

32. The medical assembly of claim 31, further comprising a releasable therapeutic substance.

33. A process for forming a medical assembly comprising cast layers of polylactic acid and alginate, wherein the alginate layer is cross-linked after casting, comprising: a) suspending a low solubility alkaline earth metal salt in a solution comprising alginate, b) casting the alginate solution on a surface, c) drying the cast alginate to form an alginate layer, d) contacting a weak acid with the alginate layer to cause the low solubility alkaline earth metal salt to dissolve within the alginate layer, thereby causing the alginate layer to crosslink.

34. The method of claim 33 wherein the alkaline earth metal salt is calcium citrate.

35. The method of claim 33 wherein the weak acid is lactic acid.

36. A method of forming a medical assembly comprising casting a solution comprising polylactic acid in a first layer, and casting a solution comprising alginate into a second layer, wherein at least one of the solutions further comprises a binding compound.

37. The method of claim 33 wherein the binding compound is selected from the group consisting of polyethylene imine, cetrimid, cationic phospholipids and any combination thereof.
